(19) United States
(12) Patent Application Publication
(10) Pub. No.: US 2022/0099645 A1
(43) Pub. Date: Mar. 31, 2022

(54) PORTABLE POISONOUS GAS MONITORING APPARATUS

(71) Applicant: TELCOKOREA IS, INC., Daejeon (KR)

(72) Inventor: Jung Jip KIM, Daejeon (KR)

(73) Assignee: TELCOKOREA IS, INC., Daejeon (KR)

(21) Appl. No.: 17/384,225

(22) Filed: Jul. 23, 2021

(30) Foreign Application Priority Data
Sep. 28, 2020 (KR) 10-2020-0125785

(51) Int. Cl. G01N 33/00; A61B 5/00; A61B 5/117; G08B 21/14; G08C 17/02

(52) U.S. Cl. CPC G01N 33/0062; A61B 5/6801; A61B 5/0002; A61B 5/117; A61B 2562/08; G08C 17/02; A61B 2503/20; A61B 2560/0242; G08B 21/14

(57) ABSTRACT

Disclosed is a portable poisonous gas monitoring apparatus including a gas sensing unit having a structure capable of being detachably attached to a work site or a worker, the gas sensing unit being configured to generate gas sensing information as the result of sensing poisonous gas, a worker sensing unit having a wearable structure capable of being worn on a portion of the body of the worker, the worker sensing unit being configured to generate life support information of the worker, a relay unit configured to receive and relay the gas sensing information and the life support information through RF communication, and a portable monitoring unit configured to receive the gas sensing information and the life support information from the relay unit through RF communication and to monitor the state of poisonous gas and the condition of the worker.

### BACKGROUND OF THE INVENTION

### Field of the Invention

The present invention relates to a portable poisonous gas monitoring apparatus.

### Description of the Related Art

In 2018, the number of people who were injured in suffocation accidents that occurred at work sites in Korea was a total of 389. Thereamong, the number of people who were injured in suffocation accidents due to leakage of and contact with chemicals was 372, and the number of people who were injured in suffocation accidents due to oxygen deficiency was 17. Suffocation accidents at work sites have steadily occurred every year, although there are slight fluctuations.

A possibility of death in suffocation disasters is much higher than other accidents, and suffocation disasters are dangerous disasters to such an extent that more than half of the injured die. In addition, there is a high possibility of two or more people simultaneously dying when accidents occur.

Suffocation disasters frequently occur in the construction industry and the manufacturing industry. In particular, the incidence rate of suffocation disasters that occur in the construction industry occupies 44% of the total incidence rate of suffocation disasters.

Therefore, there is an increasing demand for technology capable of preventing suffocation accidents of workers and guaranteeing safety of the workers.

### PRIOR ART DOCUMENT

### Patent Document

(PATENT DOCUMENT 0001) Korean Patent Application Publication No. 10-2006-0033222 (Publication Date: Apr. 19, 2006)

### SUMMARY OF THE INVENTION

It is an object of the present invention to provide a portable poisonous gas monitoring apparatus capable of preventing a suffocation accident of a worker that occurs at a work site and managing safety of the worker.

The objects of the present invention are not limited to those described above, and other unmentioned objects of the present invention will be clearly understood by those skilled in the art from the following description.

In accordance with the present invention, the above and other objects can be accomplished by the provision of a portable poisonous gas monitoring apparatus including a gas sensing unit having a structure capable of being detachably attached to a work site or a worker, the gas sensing unit being configured to generate gas sensing information as a result of sensing poisonous gas and to transmit the gas sensing information through radio frequency (RF) communication, a worker sensing unit having a wearable structure capable of being worn on a portion of a body of the worker, the worker sensing unit being configured to generate life support information of the worker and to transmit the life support information through RF communication, a relay unit configured to receive and relay the gas sensing information and the life support information transmitted respectively from the gas sensing unit and the worker sensing unit through RF communication, and a portable monitoring unit configured to receive the gas sensing information and the life support information from the relay unit through RF communication and to monitor the state of poisonous gas and the condition of the worker.

The gas sensing unit and the worker sensing unit may be located deviating from an RF communication coverage of the portable monitoring unit, and the gas sensing unit, the worker sensing unit, and the portable monitoring unit may be located within a communication coverage of the relay unit.

The gas sensing unit and the worker sensing unit may be implemented as a single body so as to be wearable on a portion of the body of the worker.

The portable monitoring unit may include a case part capable of being carried by the worker, a partition part provided in the case part, the partition part being configured to partition the interior of the case part into spaces defined to receive the gas sensing unit, the worker sensing unit, and the relay unit, a monitoring communication part provided in the case part, the monitoring communication part being configured to perform RF communication with the relay unit, a computing part provided in the case part, the computing part being configured to process the gas sensing information and the life support information received through the monitoring communication part, a display part configured to display processing results of the computing part, and a power supply part configured to supply power necessary to operate the monitoring communication part, the computing part, and the display part.

The case part may include a first body part and a second body part, the first body part and the second body part may be connected to each other via a hinge, the partition part and the computing part may be provided in the first body part, and the display part may be provided in the second body part, the display part being configured to provide the function of a touchscreen.

The gas sensing unit may be worn on a portion of the body of the worker who carries the worker sensing unit.

The portable monitoring unit may be configured to store identification information of the gas sensing unit and identification information of the worker sensing unit in a state of being matched with the worker and to display location information transmitted from at least one of the gas sensing unit and the worker sensing unit through the display part in a state of being matched with the gas sensing information and the life support information thereof.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows a portable poisonous gas monitoring apparatus according to an embodiment of the present invention;

FIG. 2 shows an example of a portable monitoring unit; and

FIG. 3 shows the internal structure of the portable monitoring unit.

### DETAILED DESCRIPTION OF THE INVENTION

Hereinafter, embodiments of the present invention will be described in detail with reference to the accompanying drawings. However, those skilled in the art will easily appreciate that the accompanying drawings are provided merely to more easily disclose the present invention and the scope of the present invention is not limited to the scope of the accompanying drawings.

In addition, the terms used in the present application are provided only to describe the specific embodiments, and do not limit the present invention. Singular forms are intended to include plural forms as well, unless the context clearly indicates otherwise.

In the present application, it should be understood that the terms “comprises,” “has,” etc. specify the presence of stated features, numbers, steps, operations, elements, components, or combinations thereof, but do not preclude the presence or addition of one or more other features, numbers, steps, operations, elements, components, or combinations thereof.

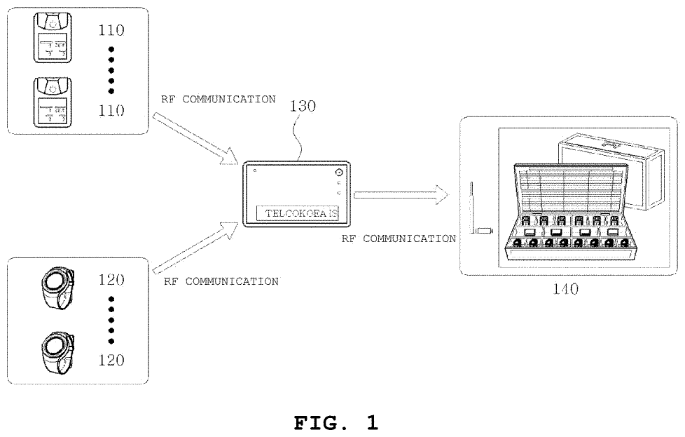

FIG. 1 shows a portable poisonous gas monitoring apparatus according to an embodiment of the present invention. As shown in FIG. 1, the portable poisonous gas monitoring apparatus according to the embodiment of the present invention includes a gas sensing unit 110, a worker sensing unit 120, a relay unit 130, and a portable monitoring unit 140.

The gas sensing unit 110 has a structure capable of being detachably attached to a work site or a worker. The gas sensing unit 110 generates gas sensing information as the result of sensing poisonous gas and transmits the gas sensing information through radio frequency (RF) communication. The gas sensing unit 110 detects poisonous gas, such as carbon monoxide, ethanol, hydrogen gas, ammonia, or methane. However, the kind of poisonous gas that is detectable is not limited thereto.

The gas sensing unit 110 may have a structure capable of being easily attached to and detached from a desired position, a portion of the body of the worker (e.g. the waist or the wrist), a work hat, a wall, a floor, or a ceiling. Such an attachable and detachable structure is well known to those skilled in the art, and therefore a description thereof will be omitted.

The gas sensing unit 110 may include an electrochemical gas sensor, an infrared gas sensor, a contact combustible gas sensor, or a semiconductor gas sensor. However, the present invention is not limited thereto.

The worker sensing unit 120 may have a wearable structure capable of being worn on a portion of the body of the worker. For example, the worker sensing unit 120 may include a band wearable on the wrist of the worker. The worker sensing unit 120 generates life support information of the worker and transmits the life support information through RF communication. The worker sensing unit 120 is provided to determine whether the worker is injured by poisonous gas and the extent of injury of the worker.

The life support information may include location information, heart rate, pulse, and oxygen saturation of the worker who wears the worker sensing unit 120. However, the present invention is not limited thereto. To this end, the worker sensing unit 120 may include a location information module, a heart rate sensor, and an oxygen saturation sensor. However, the present invention is not limited thereto.

The location information module may include a global positioning system (GPS) sensor or a signal processing module for triangulation. However, the present invention is not limited thereto.

The relay unit 130 receives the gas sensing information and the life support information transmitted respectively from the gas sensing unit 110 and the worker sensing unit 120 through RF communication. In addition, the relay unit 130 transmits the received gas sensing information and the received life support information to the portable monitoring unit 140 through RF communication.

The portable monitoring unit 140 receives the gas sensing information and the life support information from the relay unit 130 through RF communication to monitor the state of poisonous gas and the condition of the worker. For example, the portable monitoring unit 140 may provide information about the kind and concentration of poisonous gas, the position at which poisonous gas is generated, and the condition of the worker to the worker.

As described above, the gas sensing unit 110, the worker sensing unit 120, the relay unit 130, and the portable monitoring unit 140 may perform RF communication with each other. To this end, an RF communication may be included.

In order to prevent a suffocation accident due to poisonous gas at a work site, it is necessary to check the state of poisonous gas and the physical condition of the worker at the work site. However, work sites may have various different spatial structures, and it is very difficult to install a poisonous gas sensor at each work site in terms of installation cost and maintenance.

In addition, a computing device configured to process information about the state of poisonous gas and the physical condition of the worker may be spatially remote from the work site, and therefore it may be difficult for the computing device to process the information.

The portable poisonous gas monitoring apparatus according to the embodiment of the present invention includes the gas sensing unit 110, the worker sensing unit 120, and the relay unit 130, each of which is capable of being detachably attached to the work site or the worker, and the portable monitoring unit 140, which is a portable computing device. Consequently, each of the gas sensing unit 110, the worker sensing unit 120, the relay unit 130, and the portable monitoring unit 140 may include a rechargeable battery.

The worker may carry the gas sensing unit 110, the worker sensing unit 120, the relay unit 130, and the portable monitoring unit 140. The worker may install the gas sensing unit 110, the relay unit 130, and the portable monitoring unit 140 at a work site at which it is necessary to sense poisonous gas, and may wear the worker sensing unit 120, whereby it is possible to sense poisonous gas and to check the condition of the worker at various work sites.

Meanwhile, each of the gas sensing unit 110, the worker sensing unit 120, the relay unit 130, and the portable monitoring unit 140 may include an RF communication module for RF communication. The RF communication module may modulate and demodulate an RF signal.

In the present invention, communication may be performed using an RF signal having a band of 400 MHz (e.g. 447 MHz). The reason for this is that an RF signal having a band of 400 MHz is capable of being transmitted through poisonous gas and concrete of a building, whereby it is possible to smoothly monitor the state of poisonous gas and the condition of the worker.

Each of the gas sensing unit 110, the worker sensing unit 120, and the relay unit 130 may be connected to an external antenna 147 for RF communication. Alternatively, each of the gas sensing unit 110, the worker sensing unit 120, and the relay unit 130 may include an internal antenna depending on design. The external antenna 147 may be stored in a case part 141 of the portable monitoring unit 140.

The installation position of the gas sensing unit 110 may be changed depending on the kind of poisonous gas. For example, in the case in which the density of poisonous gas to be detected is higher than the density of air, the gas sensing unit 110 may be installed at the wrist of the worker, the floor, or a wall adjacent to the floor, since the poisonous gas may sink to the floor. On the other hand, in the case in which the density of poisonous gas to be detected is lower than the density of air, the gas sensing unit 110 may be installed at the head of the worker, the work hat that the worker wears, the ceiling, or a wall adjacent to the ceiling, since the poisonous gas may rise to the ceiling.

Also, in the case in which the name of poisonous gas to be detected by the worker is input, the portable monitoring unit 140 may inform the worker of the installation position or the wearing position of the gas sensing unit 110.

Meanwhile, the gas sensing unit 110 and the worker sensing unit 120 may be located deviating from an RF communication coverage of the portable monitoring unit 140, and the gas sensing unit 110, the worker sensing unit 120, and the portable monitoring unit 140 may be located within a communication coverage of the relay unit 130.

In the present invention, as previously described, the transmission and reception distance of an RF signal may be about several hundred meters when RF communication is performed. Depending on work sites, the gas sensing unit 110 and the worker sensing unit 120 may be spaced apart from the portable monitoring unit 140 by more than several hundred meters. In this case, the gas sensing information and the life support information respectively transmitted by the gas sensing unit 110 and the worker sensing unit 120 may not reach the portable monitoring unit 140.

Since the relay unit 130 relays RF communication between the gas sensing unit 110 and the portable monitoring unit 140 and between the worker sensing unit 120 and the portable monitoring unit 140, the gas sensing information and the life support information may stably reach the portable monitoring unit 140 even in the case in the distance between the gas sensing unit 110 and the portable monitoring unit 140 and between the worker sensing unit 120 and the portable monitoring unit 140 is excessively large. To this end, it is necessary for the gas sensing unit 110, the worker sensing unit 120, and the portable monitoring unit 140 to be located within the communication coverage of the relay unit 130.

Since the worker sensing unit 120 is attached to a portion of the body of the worker, the position of the worker sensing unit 120 may be changed due to movement of the worker. If some workers deviate from the communication coverage of the relay unit 130, the portable monitoring unit 140 may not receive the life support information of the workers.

The portable monitoring unit 140 may display identification information of the worker sensing unit 120, the life support information of which has not been transmitted thereto, in order to prevent a safety-related accident of the worker deviating from the communication coverage of the relay unit 130.

Meanwhile, in the case of FIG. 1, the gas sensing unit 110 and the worker sensing unit 120 are provided separately from each other. Unlike this, however, the gas sensing unit 110 and the worker sensing unit 120 may be implemented as a single body, which may be wearable on a portion of the body of the worker. Consequently, the portable monitoring unit 140 may derive gas sensing information at the position at which the worker is located. The location information of the worker was previously described above, and therefore a description thereof will be omitted.

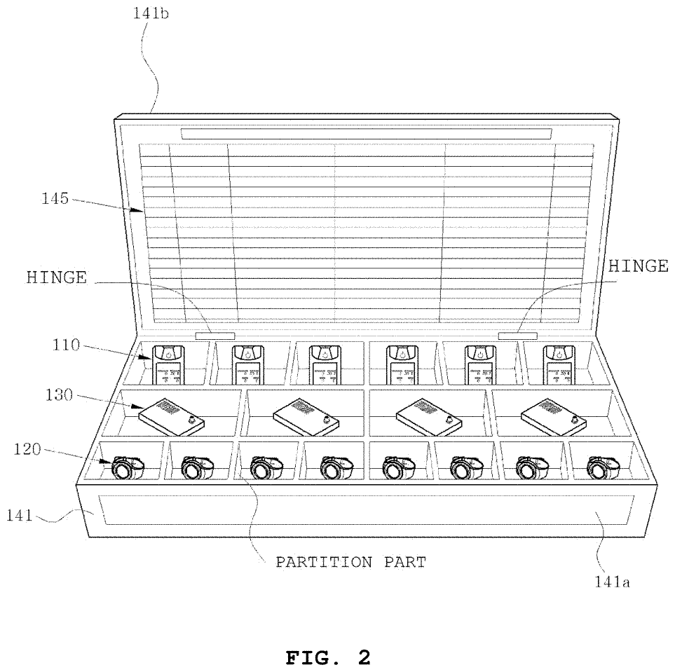

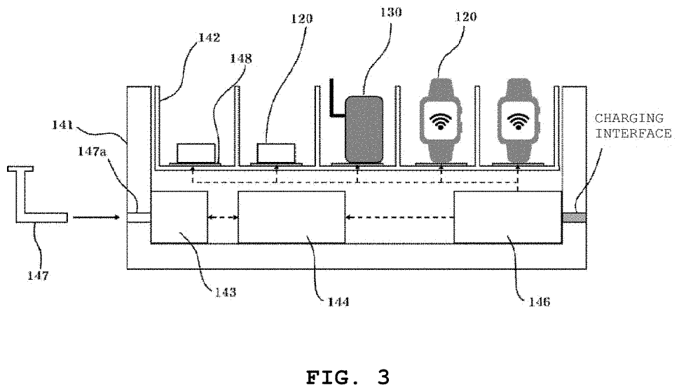

FIG. 2 shows an example of the portable monitoring unit 140. In addition, FIG. 3 shows the internal structure of the portable monitoring unit 140.

The portable monitoring unit 140 may include a case part 141, a partition part 142, a monitoring communication part 143, a computing part 144, a display part 145, and a power supply part 146.

The case part 141 may be carried by the worker. The case part 141 may be easy to carry and may protect various instruments or parts disposed therein from an external environment.

The partition part 142 is provided in the case part 141 to partition the interior of the case part into spaces defined to receive the gas sensing unit 110, the worker sensing unit 120, and the relay unit 130. For example, as shown in FIG. 3, the partition part 142 may provide a space in which each of the gas sensing unit 110, the worker sensing unit 120, and the relay unit 130 can be disposed.

In FIG. 3, a plurality of side surfaces of the partition part 142 that defines the spaces is formed perpendicular to the bottom surface of the partition part 142. However, this is merely an example. For example, the partition part 142 may provide concave spaces corresponding to the outer shapes of the gas sensing unit 110, the worker sensing unit 120, and the relay unit 130.

The monitoring communication part 143 may be provided in the case part 141 in order to perform RF communication with the relay unit 130.

The monitoring communication part 143 may have a built-in antenna. In the case in which the case part 141 is made of a metallic material, such as aluminum, however, the metallic material may shield an RF signal of the relay unit.

For this reason, as shown in FIG. 3, an external antenna 147 may be provided. The external antenna 147 may be connected to or disconnected from the monitoring communication part 143. Since the external antenna 147 may be located outside the case part, the external antenna 147 may be inserted into a connection hole 147a formed in the outer surface of the case part 141 so as to be connected to the monitoring communication part 143.

Although not shown in FIG. 3, the partition part 142 may also provide a space configured to store the external antenna 147.

The computing part 144 may be provided in the case part 141 in order to process the gas sensing information and the life support information received through the monitoring communication part 143. To this end, the computing part 144 may include a memory and a processor. The processor may be a dedicated controller or a universal CPU. However, the present invention is not limited thereto.

The memory may store identification information of gas sensing units 110, worker sensing units 120, and relay units 130, identification information of workers, gas sensing information for each gas sensing unit 110, and life support information for each worker sensing unit 120. The identification information of the worker sensing units 120 and the identification information of the workers may be stored in a state of being matched with each other. However, information stored in the memory is not limited thereto.

Processing results of the computing part 144 may be various. For example, the kind and concentration of poisonous gas and the heart rate and oxygen saturation of each worker may be derived for each zone of a work site.

The display part 145 may display the processing results of the computing part 144. The display part 145 may be implemented by an LCD or an OLED. However, the present invention is not limited as to such display devices.

As shown in FIG. 2, the case part 141 may include a first body part 141a and a second body part 141b. The first body part 141a and the second body part 141b may be connected to each other via a hinge. The partition part 142 and the computing part 144 may be provided in the first body part 141a, and the display part 145 may be provided in the second body part 141b. In addition, the display part 145 may provide the function of a touchscreen.

The portable monitoring unit 140 of the present invention may simultaneously perform a function of storing the gas sensing unit 110, the worker sensing unit 120, and the relay unit 130 and a function of processing the gas sensing information and the life support information.

Consequently, the display part 145 may also be provided in the portable monitoring unit 140. In the case in which various kinds of processing information are seen when the worker opens the second body part 141b of the portable monitoring unit 140, worker convenience may be improved. For this reason, the display part 145 may be provided in the second body part 141b.

In the present invention, a keyboard or mouse necessary to input various kinds of information may be included. In this case, the keyboard or the mouse may be stored in the portable monitoring unit 140 and may be connected to the computing part 144 whenever used, which is inconvenient.

In the present invention, the display part 145 that provides the function of a touchscreen may be included, whereby it is possible to improve worker convenience in inputting of information.

Meanwhile, the power supply part 146 may supply power necessary to operate the monitoring communication part 143, the computing part 144, and the display part 145. The power supply part 146 may directly supply voltage suitable for each of the monitoring communication part 143, the computing part 144, and the display part 145. Alternatively, the computing part 144 may receive power from the power supply part 146 and may supply voltage suitable for each of the monitoring communication part 143, the computing part 144, and the display part 145.

Since the portable monitoring unit 140 may be carried by the worker, the power supply part 146 may include a battery, a charging circuit, and an overcharging prevention circuit. However, the present invention is not limited thereto. The charging circuit may support quick charging of the battery. A charging interface (e.g. a USB interface) configured to charge the power supply part 146 may be provided at a side surface of the case part.

Meanwhile, the power supply part 146 or the computing part 144 may supply power to a wireless charging pad 148 configured to wirelessly charge the gas sensing unit 110, the worker sensing unit 120, and the relay unit 130. The wireless charging pad 148 may be installed at the bottom of the partition part 142 in contact with the gas sensing unit 110, the worker sensing unit 120, and the relay unit 130. In addition, each of the gas sensing unit 110, the worker sensing unit 120, and the relay unit 130 may be provided with a power receiving part configured to wirelessly receive power from the charging pad.

For the wireless charge, each of the wireless charging pad 148 and the power receiving part may include a coil.

As previously described, the gas sensing units 110, the worker sensing units 120, and the relay units 130 may be installed at various work sites and workers, and may be stored in the portable monitoring unit 140 after use thereof.

Consequently, each of the gas sensing units 110, the worker sensing units 120, and the relay units 130 may include a battery, and the battery needs to be charged. A plurality of gas sensing units 110, a plurality of worker sensing units 120, and a plurality of relay units 130 must be charged. In the case in which wired charging is performed, unlike the present invention, the plurality of gas sensing units 110, the plurality of worker sensing units 120, and the plurality of relay units 130 may be connected in a wired manner, which is troublesome.

Also, in the case in which the portable monitoring unit 140 is provided with neither a wired charging circuit nor a wireless charging circuit, the plurality of gas sensing units 110, the plurality of worker sensing units 120, and the plurality of relay units 130 may be taken out of the portable monitoring unit 140 for charging, which is inconvenient.

In the present invention, the wireless charging pad 148 is provided at the bottom of the partition part 142, whereby charging is possible simply by inserting each of the gas sensing unit 110, the worker sensing unit 120, and the relay unit 130 into the partition part 142, and therefore a charging task may be conveniently performed by the worker.

As previously described, the gas sensing unit 110 may be wearable on a portion of the body of the worker who carries the worker sensing unit 120. In this case, the portable monitoring unit 140 may store the identification information of the gas sensing unit 110 and the identification information of the worker sensing unit 120 in a state of being matched with the worker.

At this time, the portable monitoring unit 140 may display location information transmitted from at least one of the gas sensing unit 110 and the worker sensing unit 120 through the display part 145 in a state of being matched with the gas sensing information and the life support information thereof.

For example, the display part 145 may display a map of the current work site and may collectively display the location of the worker, the physical condition of the worker, the kind of poisonous gas at the position at which the worker is located, and the concentration of poisonous gas at the position at which the worker is located on the map.

As is apparent from the above description, the portable poisonous gas monitoring apparatus according to the embodiment of the present invention is capable of measuring poisonous gas generated at a work site and informing a worker of the safety state of the work site stepwise (safety/caution/warning) such that the worker can rapidly escape and take a proper measure, whereby it is possible to prevent or minimize a personal accident due to the poisonous gas.

The effects of the present invention are not limited to those mentioned above, and other unmentioned effects will be clearly understood by those skilled in the art from the above description.

While the embodiments of the present invention have been described with reference to the accompanying drawings, those skilled in the art will appreciate that the present invention may be embodied in specific forms other than those set forth herein without departing from the gist and category of the present invention. The disclosed embodiments are therefore to be construed as illustrative and not restrictive. Consequently, the present invention is not limited to the above description, and may be changed within the category of the appended claims and an equivalent scope thereof.

What is claimed is:

1. A portable poisonous gas monitoring apparatus comprising:
a gas sensing unit having a structure capable of being detachably attached to a work site or a worker, the gas sensing unit being configured to generate gas sensing information as a result of sensing poisonous gas and to transmit the gas sensing information through radio frequency (RF) communication;
a worker sensing unit having a wearable structure capable of being worn on a portion of a body of the worker, the worker sensing unit being configured to generate life support information of the worker and to transmit the life support information through RF communication;
a relay unit configured to receive and relay the gas sensing information and the life support information transmitted respectively from the gas sensing unit and the worker sensing unit through RF communication; and
a portable monitoring unit configured to receive the gas sensing information and the life support information from the relay unit through RF communication and to monitor a state of poisonous gas and a condition of the worker.

2. The portable poisonous gas monitoring apparatus according to claim 1, wherein
the gas sensing unit and the worker sensing unit are located deviating from an RF communication coverage of the portable monitoring unit, and
the gas sensing unit, the worker sensing unit, and the portable monitoring unit are located within a communication coverage of the relay unit.

3. The portable poisonous gas monitoring apparatus according to claim 1, wherein the gas sensing unit and the worker sensing unit are implemented as a single body so as to be wearable on a portion of the body of the worker.

4. The portable poisonous gas monitoring apparatus according to claim 1, wherein the portable monitoring unit comprises:
a case part capable of being carried by the worker;
a partition part provided in the case part, the partition part being configured to partition an interior of the case part into spaces defined to receive the gas sensing unit, the worker sensing unit, and the relay unit;
a monitoring communication part provided in the case part, the monitoring communication part being configured to perform RF communication with the relay unit;
a computing part provided in the case part, the computing part being configured to process the gas sensing information and the life support information received through the monitoring communication part;
a display part configured to display processing results of the computing part; and
a power supply part configured to supply power necessary to operate the monitoring communication part, the computing part, and the display part.

5. The portable poisonous gas monitoring apparatus according to claim 4, wherein
the case part comprises a first body part and a second body part,
the first body part and the second body part are connected to each other via a hinge,
the partition part and the computing part are provided in the first body part, and
the display part is provided in the second body part, the display part being configured to provide a function of a touchscreen.

6. The portable poisonous gas monitoring apparatus according to claim 1, wherein the gas sensing unit is configured to be worn on a portion of the body of the worker who carries the worker sensing unit.

7. The portable poisonous gas monitoring apparatus according to claim 6, wherein the portable monitoring unit is configured:
to store identification information of the gas sensing unit and identification information of the worker sensing unit in a state of being matched with the worker; and
to display location information transmitted from at least one of the gas sensing unit and the worker sensing unit through the display part in a state of being matched with the gas sensing information and the life support information thereof.
